(19) United States
(12) Patent Application Publication
(10) Pub. No.: US 2017/0224750 A1
(43) Pub. Date: Aug. 10, 2017

(54) LACTOBACILLUS PLANTARUM CNCM I-4026 PREPARATIONS AND SKIN HEALTH

(71) Applicant: NESTEC S.A., Vevey (CH)

(72) Inventors: Michael John Callanan, Lausanne (CH); Olga Sakwinska, Lausanne (CH); Gilles Bourdin, Fribourg (CH); Stephane Duboux, St-Prex (CH); Edwin Ananta, Epalinges (CH)

(21) Appl. No.: 15/503,164

(22) PCT Filed: Jul. 8, 2015
(86) PCT No.: PCT/EP2015/065602

§ 371 Date: Feb. 10, 2017

(30) Foreign Application Priority Data
Aug. 13, 2014 (EP) 14180898.0

(51) Int. Cl. A61K 35/747; A61K 45/06; A61K 8/06; A61Q 17/00; A61Q 19/00; A61K 9/00; A61K 8/99

(52) U.S. Cl. CPC A61K 35/747; A61K 9/0014; A61K 9/0017; A61K 45/06; A61K 8/99; A61Q 17/005; A61Q 19/00; A61K 8/062; A61K 2800/70

(57) ABSTRACT

The present invention relates generally to compositions with a health benefit. In particular, the invention relates to bacterial preparations of Lactobacillus plantarum CNCM I-4026 (NCC 2936). An aspect of the invention is a composition for use in the treatment or prevention of skin infections comprising a bacterial preparation of Lactobacillus plantarum CNCM I-4026. A further aspect is a cosmetic composition comprising a bacterial preparation of Lactobacillus plantarum CNCM I-4026.

The present invention relates generally to compositions with a health benefit. In particular, the invention relates to bacterial preparations of Lactobacillus plantarum CNCM I-4026 (NCC 2936). An aspect of the invention is a composition for use in the treatment or prevention of skin infections comprising a bacterial preparation of Lactobacillus plantarum CNCM I-4026. A further aspect is a cosmetic composition comprising a bacterial preparation of Lactobacillus plantarum CNCM I-4026.

Antimicrobial agents have played a key role in improving public health in modern society. They find many diverse applications such as disinfecting surfaces, preserving food or as therapeutic agents in healthcare. One application of antimicrobial agents is in skin health, for example, as a disinfectant hand wash, or as an antibiotic ointment applied to the skin to treat skin infections.

The skin is the largest organ of the human body. It is our first line of defense against the environment, protecting the body against pathogenic micro-organisms and preventing excessive water loss. Because of its structural organization and the wide variety of cells it is made of, the skin ensures a large spectrum of biological functions including wound healing, immune response, body temperature regulation and vitamin D production. Although many bacteria come in contact with or reside on the skin, they are normally unable to establish an infection. However, the risk of getting a bacterial skin infection becomes much higher when the skin is broken. Many types of bacteria can infect the skin. Some of the most common types of skin infection are staphylococcal infections.

Some people are at particular risk of developing skin infections. For example, people with diabetes are likely to have poor blood flow, especially to the hands and feet, and the high levels of sugar (glucose) in their blood decrease the ability of white blood cells to fight infections. People who are older, who have human immunodeficiency virus (HIV) or other immune disorders, or hepatitis, and who are undergoing chemotherapy or treatment with other drugs that suppress the immune system are at higher risk as well because they have a weakened immune system [The Merck Manual Home Health Handbook—3rd Edition]. Skin that is inflamed or damaged by sunburn, scratching, or other trauma is more likely to become infected.

Oral antibiotics may be used against skin infections, especially serious cases. Topical antibiotics are also widely used, for example combinations of antibiotics such as bacitracin, neomycin and polymyxin B. Some people have allergic reactions to certain antibiotics, and widespread use of an antibiotic may lead to the emergence of antibiotic resistant bacteria. It would therefore be valuable to be able to provide alternative antibiotic compositions.

Sanitizing hand washes are used by many professions such as food handlers and medical care workers. As well as preventing transfer of harmful bacteria, they can protect the user from skin infections. Alcohol-based hand washes are generally effective against bacteria, but repeated use may lead to dry skin. Dry skin easily becomes damaged, and may provide an entry point for bacteria which cause skin infections. It would be advantageous to have a hand wash with antibiotic properties where the active ingredient does not promote dry skin. Many users would also prefer to apply products with active ingredients obtained from natural sources.

Lactic acid bacteria provide a promising source of antimicrobial activity for use in health care as they have a long and safe tradition in food fermentation processes.

However, most lactic acid bacteria exhibit antimicrobial activity against only a narrow range of microorganisms. There is a need to provide antimicrobial agents from natural sources which show efficacy against a wide range of microorganisms, in particular, both Gram-positive and Gram-negative bacteria. For use in treating or preventing skin infections it is valuable to be able to provide antimicrobial agents which are effective against staphylococcal infections. A persistent problem in providing effective antimicrobial agents is that pathogenic bacteria develop resistance to existing antimicrobial compounds over time. There is therefore an ongoing need for the development of new antimicrobial compositions in order to continually meet the challenges presented by resistant strains.

An object of the present invention is to improve the state of the art and to provide alternative antimicrobial agents from a natural source that can be used in the treatment or prevention of skin infections. Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”. The object of the present invention is achieved by the subject matter of the independent claim. The dependent claims further develop the idea of the present invention.

The inventors were surprised to find that bacterial preparations of Lactobacillus plantarum CNCM I-4026 have wide-ranging antimicrobial properties. For example, the fermentation broth resulting after fermentation of Lactobacillus plantarum CNCM I-4026 was found to inhibit or delay the growth of a number of different microorganisms. The microorganisms delayed or inhibited by the broth included Staphylococcus aureus which is commonly associated with skin infections, as well as Staphylococus epidermidis which is also associated with skin infections, particularly in patients with compromised immune systems. The broth also was able to delay or inhibit the growth of Escherichia coli which may be found on skin due to poor hygiene practices and can cause or complicate infections of the skin. The fermentation broth resulting after fermentation of Lactobacillus plantarum CNCM I-4026 was found to, for example, inhibit or delay growth of both Gram-positive and Gram-negative bacteria. Although antimicrobial activity has been shown for other Lactobacillus plantarum strains, it surprising that a single Lactobacillus plantarum strain can inhibit such a wide range of bacteria. Lactobacillus plantarum CNCM I-4026 was found to be more effective than Lactobacillus plantarum strains with known antimicrobial properties at limiting the growth of Staphylococcus aureus and other harmful bacteria.

Bacterial preparations of Lactobacillus plantarum CNCM I-4026 have surprisingly been found to inhibit or delay the growth of organisms associated with skin infections. Accordingly, an embodiment of the invention is a composition for use in the treatment or prevention of skin infections comprising a bacterial preparation of Lactobacillus plantarum CNCM I-4026. A further aspect is a cosmetic composition comprising a bacterial preparation of Lactobacillus plantarum CNCM I-4026 wherein the composition is selected from the group consisting of cleansing, protective or care creams, milks or lotions for the face, for the hands, for the feet, for the major anatomical folds or for the body; makeup products; skincare gels or foams; shampoos, soaps; bath compositions; deodorant compositions, aftershave gels or lotions; and depilatory creams.

Lactobacillus plantarum NCC 2936 is a sialic acid producing bacteria which previously was only known for its use in enriching the nutritional value of food by providing a source of sialic acid (EP2183984). Lactobacillus plantarum NCC 2936 was deposited with the Collection Nationale de Cultures de Microorganismes (CNCM), Institut Pasteur, 25 rue du Docteur Roux, F-75724 PARIS Cedex 15, France, on 25 Jun. 2008 and given the deposit number CNCM I-4026.

The present invention relates to a composition for use in the treatment or prevention of skin infections comprising a bacterial preparation of Lactobacillus plantarum CNCM I-4026 (NCC 2936). A bacterial preparation of Lactobacillus plantarum CNCM I-4026 has wide-ranging antimicrobial properties including the ability to inhibit or delay the growth of bacteria which cause or complicate skin infections. The bacterial preparation of the composition for use in the present invention may be applied topically, for example it may be applied directly to the skin. The bacterial preparation of the composition for use in the present invention may comprise live Lactobacillus plantarum CNCM I-4026 bacteria. These live bacteria compete with harmful bacteria on the skin, further improving skin health. The bacterial preparation of the present invention may comprise an aqueous suspension of bacteria, for example bacteria growing in a liquid nutritional medium.

The quantity of bacterial preparation in the composition for use in the present invention may be an amount that is sufficient to obtain the expected effect (an “effective amount”). The composition for use in the present invention may contain between 0.05 and 20 wt. % of bacterial preparation, the weight of bacterial preparation being on a dry basis. Quantities of the bacterial preparation above 10 wt. % may be required for preparations to treat infected wounds in the skin, whereas preparations to treat more minor skin infections may have lower quantities of the bacterial preparation. For example the composition for use in the present invention may contain between 0.1 and 10 wt. % of bacterial preparation, the weight of bacterial preparation being on a dry basis.

The bacterial preparation may be a fermentation broth of Lactobacillus plantarum CNCM I-4026 or an extract thereof. The term fermentation broth refers to the culture medium resulting after fermentation of bacteria, including the bacteria and/or its component parts; unused raw substrates; and metabolites produced by the bacteria during fermentation. The fermentation broth may be a concentrated or dehydrated fermentation broth. Use of a fermentation broth is advantageous as the metabolites in the broth following fermentation with Lactobacillus plantarum CNCM I-4026 include a range of different materials with antimicrobial activities. For example the fermentation broth may contain low molecular weight organic acids, bacteriocins and antifungal peptides which have a beneficial antimicrobial effect. Preferably the fermentation broth is suitable for consumption by humans or pets in case of inadvertent ingestion of the composition.

The bacterial preparation may be a fermentation broth of Lactobacillus plantarum CNCM I-4026 wherein the Lactobacillus plantarum CNCM I-4026 have been rendered non-viable, or an extract of a fermentation broth of Lactobacillus plantarum CNCM I-4026 wherein the Lactobacillus plantarum CNCM I-4026 have been rendered non-viable. The Lactobacillus plantarum CNCM I-4026 in the fermentation broth may have been rendered non-viable by any method known in the art. For example, the Lactobacillus plantarum CNCM I-4026 may be rendered non-viable by heat treatment, sonification, microwave irradiation, gamma irradiation, ultraviolet irradiation, mechanical cell disruption, chemical cell disruption or combinations thereof. Compositions which do not contain viable bacteria are easier to store and to introduce into products.

In the present invention, bacteria are described as non-viable when no viable cells and/or colony forming units can be detected by classical plating methods. Such classical plating methods are summarized in the microbiology book: James Monroe Jay, Martin J. Loessner, David A. Golden. 2005. Modern food microbiology. 7th edition, Springer Science, New York, N.Y. 790 p. Typically, the absence of viable cells can be shown as follows: no visible colony on agar plates or no increasing turbidity in liquid growth medium after inoculation and incubation under appropriate conditions (aerobic and/or anaerobic atmosphere for at least 24 h). In the bacterial preparation comprised within the composition of the present invention, at least 80%, preferably at least 90%, more preferably at least 95%, ideally at least 99.9% of the Lactobacillus plantarum CNCM I-4026 may be non-viable. The bacterial preparation may have a viable cell count of less than 1000 colony forming units (CFU) per ml, for example less than 200 CFU/ml, for further example less than 10 CFU/ml.

The bacterial preparation may be an extract of a fermentation broth of Lactobacillus plantarum CNCM I-4026. For example the Lactobacillus plantarum CNCM I-4026 may have been at least partially removed from the fermentation broth. Preferably the fermentation broth before the at least partial removal of the Lactobacillus plantarum CNCM I-4026 contains between 10⁵ and 10¹¹ colony forming units (CFU) per ml of Lactobacillus plantarum CNCM I-4026. In the case of Lactobacillus plantarum CNCM I-4026 which have been rendered non-viable, this term is to be understood as the amount of non-viable bacteria that is obtained from 10⁵ and 10¹¹ CFU/ml viable bacteria. In the present invention, “at least partially removed” means that at least 50% of the Lactobacillus plantarum CNCM I-4026 cells present in the bacterial preparation have been removed, for example at least 80% of the Lactobacillus plantarum CNCM I-4026 cells, or at least 90% of the Lactobacillus plantarum CNCM I-4026 cells, or at least 99.9%. For example all Lactobacillus plantarum CNCM I-4026 cells may have been removed. The cells may be at least partially removed by filtration and/or centrifugation.

The bacterial preparation may be the supernatant of a fermentation broth of Lactobacillus plantarum CNCM I-4026 after centrifugation. The supernatant of a fermentation broth of Lactobacillus plantarum CNCM I-4026 after centrifugation may have a cell count of less than 2000 CFU/ml. Such supernatants or filtrates may be concentrated or fractionated and may be dried to a powder. The bacterial preparation may be obtained as a by-product from the production of Lactobacillus plantarum CNCM I-4026, for example the production of Lactobacillus plantarum CNCM I-4026 for incorporation in a foodstuff as a source of sialic acid. Commonly in the production of bacteria, once the majority of bacterial cells have been removed or “harvested” from a fermentation broth, the rest of the broth is discarded. This is wasteful and it is often costly to ensure a safe and environmentally responsible disposal of the remaining broth. It is therefore an advantage that this by-product can be beneficially used in the current invention. The bacterial preparation may be obtained, for example obtainable, by a process comprising the steps of preparing a fermentation broth which contains between 10⁵ and 10¹¹ colony forming units per ml of Lactobacillus plantarum CNCM I-4026, removing substantially all the cells and concentrating the broth. The step of removing substantially all the cells may be performed by centrifugation and/or sterile filtration.

The composition of the invention may be for use in the treatment or prevention of skin infections selected from the group consisting of boils, carbuncles, styes, impetigo, cellulitis, acne vulgaris and combinations of these. A boil, also called a furuncle, is a deep folliculitis, infection of the hair follicle. It is most commonly caused by infection by the bacterium Staphylococcus aureus, resulting in a painful swollen area on the skin caused by an accumulation of pus and dead tissue. Individual boils clustered together are called carbuncles. An external stye or hordeolum is an infection of the sebaceous glands at the base of the eyelashes, or an infection of the apocrine sweat glands. External styes form on the outside of the eyelids and can be seen as small red bumps. Internal styes are infections of the meibomian sebaceous glands lining the inside of the eyelids. Styes are usually caused by the Staphylococcus aureus bacterium.

Impetigo is a common and highly contagious bacterial skin infection that causes sores and blisters. It is primarily caused by Staphylococcus aureus, and sometimes by Streptococcus pyogenes.

Cellulitis is an acute bacterial infection of the skin and subcutaneous tissue most often caused by Streptococcus pyogenes or Staphylococcus aureus. Symptoms and signs are pain, rapidly spreading erythema, and edema; fever may occur, and regional lymph nodes may enlarge.

Acne vulgaris (acne) is the formation of comedones, papules, pustules, nodules, and/or cysts as a result of obstruction and inflammation of pilosebaceous units (hair follicles and their accompanying sebaceous gland). It most often affects adolescents. Propionibacterium acnes, Staphylococcus epidermidis and Staphylococcus aureus have all been implicated in causing acne [F. Khorvash et al., N Am J Med Sci., 4(11), 573-576 (2012)].

Staphylococcal infections are a group of infections caused by the bacterium Staphylococcus. There are many types of staphylococci, but most skin infections are caused by Staphylococcus aureus which is common, and often found in the nose or on the skin. A bacterial preparation of Lactobacillus plantarum CNCM I-4026 was found to be effective at limiting the growth of Staphylococcus aureus. The composition of the invention may be for use in the treatment or prevention of skin infections wherein the skin infections are staphylococcal infections.

Pathogenic bacteria develop resistance to existing antimicrobial compounds over time. For example, methicillin-resistant Staphylococcus aureus (MRSA), also called oxacillin-resistant Staphylococcus aureus (ORSA), is a bacterium responsible for several difficult-to-treat infections in humans and animals. It is therefore beneficial to provide new antimicrobial compositions. The composition of the invention may be for use in the treatment or prevention of skin infections wherein the skin infections are methicillin-resistant Staphylococcus aureus infections.

Staphylococcal skin infections are common, particularly among children, teenagers and young adults. Impetigo, for example, accounts for around 1 in 10 of all reported skin conditions in children. The composition for use according to the invention may be administered to a child, teenager or young adult. In the context of the present invention a child is a human between the stages of birth and puberty, a teenager is aged between 13 and 19 inclusive, and a young adult is aged between 20 and 40.

People or animals that already have damaged skin are particularly susceptible to skin infections as the damage provides an entry point for the bacteria. In addition, an underlying trauma or disease may affect the general health of the subject and reduce their natural immune defences. The composition for use according to the invention may be administered to a subject suffering from burns, cuts, abrasions, pressure ulcers or combinations of these.

Atopic dermatitis, also known as atopic eczema or eczema, is an inflammatory, relapsing, non-contagious and itchy skin disorder. Staphylococcus aureus may colonize the skin of patients with atopic dermatitis, and is consistently found in eczematous skin lesions in these patients. A correlation between the severity of the eczema and colonization with Staphylococcus aureus has been demonstrated, and it has been determined that bacterial colonization is an important factor aggravating skin lesions. The composition for use according to the invention may be administered to a subject suffering from atopic dermatitis.

Combining different antimicrobial compounds can lead to improved effectiveness. This may be due to different bacteria having different susceptibilities to the different antimicrobials and also to bacteria being weakened by one antibacterial agent and then killed by a second agent in a “combined attack”. As bacterial preparations of Lactobacillus plantarum CNCM I-4026 would be desirable to many consumers due to being from a natural source, it is advantageous to combine bacterial preparations of Lactobacillus plantarum CNCM I-4026 with other natural antimicrobial compounds.

The composition for use according to the invention may further comprise a compound with antimicrobial properties selected from the group consisting of tea tree oil, eugenol, thymol, carvacrol, cinnamaldehyde, allyl isothiocyanate, lauric acid, palmitoleic acid, linoleic acid, acetic acid, propionic acid, butyric acid and combinations of these.

Bacterial preparations of Lactobacillus plantarum CNCM I-4026 may be mixed with other ingredients to provide a convenient dosage form. The composition for use according to the invention may be selected from the group consisting of a lotion (for example a body lotion or hand lotion), skin cream, ointment, gel (for example a semi-solid emulsion in an alcohol base), foam, shampoo and hand wash. The composition may be applied at a dosage of between 1 and 200 mg of the bacterial preparation of Lactobacillus plantarum CNCM I-4026 on a dry basis per 100 cm² of skin, 1 to 4 times per day. For example, the composition may be applied at a dosage of between 5 and 100 mg of the bacterial preparation of Lactobacillus plantarum CNCM I-4026 on a dry basis per 100 cm² of skin, 1 to 4 times per day.

In dogs and cats, Staphylococcus bacteria most commonly causes skin infections like folliculitis, furunculosis, boils, and cellulitis. In dogs and cats, Staphylococcus infections are usually secondary infections. The most common reason for recurring or chronic Staphylococcus infections in dogs is allergic skin disease. It is advantageous to be able to provide a composition which can inhibit or delay the growth of organisms associated with skin infections, in particular a composition which is safe to be ingested, as it is often not possible to prevent pets from licking their infected skin. The composition for use according to the invention may be a pet care product.

In a further aspect, the invention provides a cosmetic composition comprising a bacterial preparation of Lactobacillus plantarum CNCM I-4026 wherein the composition is selected from the group consisting of cleansing, protective or care creams, milks or lotions for the face, for the hands, for the feet, for the major anatomical folds or for the body (for example, day creams, night creams, makeup-removing creams, sun creams, after-sun creams); makeup products (such as fluid foundations and makeup-removers); skincare gels or foams; shampoos (for example antidandruff shampoos), soaps; bath compositions; deodorant compositions, aftershave gels or lotions; and depilatory creams. These cosmetic compositions are prepared according to the usual methods. For example the cosmetic composition comprising a bacterial preparation of Lactobacillus plantarum CNCM I-4026 may be selected from the group consisting of lotions, skin creams, shampoos, soaps and deodorants.

A cosmetic composition has a cosmetic function, for example altering or improving appearance, but the incorporation of a bacterial preparation of Lactobacillus plantarum CNCM I-4026 provides an additional antimicrobial effect. For example, a coloured cosmetic skin cream for concealing skin blemishes such as acne spots or blackheads may also contain a bacterial preparation of Lactobacillus plantarum CNCM I-4026 as an antimicrobial ingredient to help clear up the blemishes. The addition of a bacterial preparation of Lactobacillus plantarum CNCM I-4026 to a deodorant or soap helps reduce the number of odour-causing bacteria. The invention may provide cosmetic use of a composition comprising a bacterial preparation of Lactobacillus plantarum CNCM I-4026. A bacterial preparation of Lactobacillus plantarum CNCM I-4026 may also be comprised within a cosmetic composition as a preservative to prevent the growth of spoilage organisms. The quantity of bacterial preparation in the cosmetic composition of the invention may be an amount that is sufficient to obtain the expected effect (an “effective amount”). The amount of the bacterial preparation of Lactobacillus plantarum CNCM I-4026 in the cosmetic composition of the invention may be between 0.05 and 20 wt. %, the weight of bacterial preparation being on a dry basis.

The cosmetic composition may further comprise a compound with antimicrobial properties selected from the group consisting of tea tree oil, eugenol, thymol, carvacrol, cinnamaldehyde, allyl isothiocyanate, lauric acid, palmitoleic acid, linoleic acid, acetic acid, propionic acid, butyric acid and combinations of these.

Those skilled in the art will understand that they can freely combine all features of the present invention disclosed herein. In particular, features described for different embodiments of the present invention may be combined. Where known equivalents exist to specific features, such equivalents are incorporated as if specifically referred to in this specification. Further advantages and features of the present invention are apparent from the non-limiting examples.

### EXAMPLES

### Example 1: Lactobacillus plantarum NCC2936 Antimicrobial Activity

A vial of L. plantarum CNCM I-4026 was reactivated in 10 ml MRS broth (Difco) and incubated overnight at 30° C. in sterile anaerobic conditions (1st culture). A second culture was made by inoculating 10m1 of MRS broth with 2% of the previous culture (2nd culture). An inoculum was then prepared in 250 ml of MRS broth, inoculated at 2% with the 2nd culture. The final fermentation was performed in a 1 litre bioreactor containing MRS broth. Following the fermentation, the culture was centrifuged for 20 minutes at 3300×g (gravities) in sterile 1 L plastic bottles. The resulting pellet was discarded and the supernatant was filtered through a 0.2 μm filter. The filtered supernatant was frozen at −40° C. in a sealed aluminium bag and freeze-dried using a LyoBeta 35 instrument. The resulting powder was stored at 4° C. and rehydrated in the appropriate media for the inhibition assays.

Strains of typical microorganisms were collected from a variety of sources. In general, the selected strains were environmental isolates from food products and clinical sources. The strains included both clinical and food isolates of Staphylococcus aureus, together with an isolate of Staphylococus epidermidis. To perform the inhibition assays, cultures were grown overnight using appropriate media. Overnight cultures were diluted to achieve a starting inoculum of between 10³ and 10⁴ CFU/ml in normal growth media or media adjusted to pH 5.5. The freeze-dried filtered supernatant described above was rehydrated at 5% (w/v) in the growth media to form an antimicrobial stock solution. In standard multiwell plates, 20 μl of this stock antimicrobial solution was added to the diluted culture to give a final volume of 200 μl per well and a final concentration of 0.5% of the freeze dried material. The plate was loaded in a Bioscreen C Microbiological Analyser (Labsystems Helsinki, Finland) and incubated at 30° C. or 37° C. with shaking for 24-48 hours. The Optical Density (OD) was recorded every 15 minutes at 600 nm.

A delay or failure to record the Time To Detection (TTD) for each culture in the presence of the antimicrobial compared to the control was taken as the measure of inhibition. The TTD was determined when the OD₆₀₀ reading increased by 0.2 above the background for the media plus the antimicrobial. A delay was recorded when a slower TTD relative to the control was observed and this difference could vary considerably but generally required a minimum change of 2 hours. All assays were performed in triplicate. The results are presented in tables 1 and 2 below.

The results show that the bacterial preparation of Lactobacillus plantarum CNCM I-4026 has surprisingly wide-ranging antimicrobial properties; inhibiting or delaying the growth of many microorganisms associated with poor health and in particular Staphylococcus aureus, Staphylococus epidermidis and Escherichia coli which may cause or complicate skin infections. The bacterial preparation of Lactobacillus plantarum CNCM I-4026 is shown to inhibit or delay growth of both Gram-positive and Gram-negative bacteria.

TABLE 1
Inhibition assay results for NCC2936 supernatant-Food safety/spoilage
microorganisms.
Food safety/spoilage microorganism

| Gram Positive | | Gram Negative | |
|---|---|---|---|
| Bacillus cereus | I | Escherichia coli | I |
| Bacillus licheniformis | I | Pseudomonas fluorescens | I |
| Lactobacillus plantarum | X | Salmonella enteriditis | D |
| Enterococcus faecium | I | Enterobacter cloacae | D |
| Staphylococcus aureus (food source) | I | | |
| Listeria monocytogenes | I | | |

I = No TTD determined, X = Growth detected and D = Delay in TTD.

TABLE 2
Inhibition assay results for NCC2936 supernatant-Respiratory/oral
microorganisms.
Respiratory/oral microorganism

| Gram Positive | Gram Negative | | |
|---|---|---|---|
| Streptococcus pneumoniae | I | Haemophilus influenzae | I |
| Streptococcus pseudopneumoniae | I | Haemophilus parainfluenzae | I |
| Streptococcus mitis | I | Pseudomonas aeruginosa | I |
| Streptococcus oralis | I | | |
| Staphylococcus aureus (clinical isolate) | I | | |
| Staphylococus epidermidis | I | | |

I = No TTD determined, X = Growth detected and D = Delay in TTD.

### Example 2: Comparison of Lactobacillus plantarum Strains

Three plantarum strains were compared for their antimicrobial activity; L. plantarum CNCM I-4026, L. plantarum ATCC 8014 and L. plantarum WCFS1. L. plantarum ATCC 8014 has been shown to display antimicrobial properties [B. W. Lash et al., Food Microbiology 22, 199-204 (2005)] [N. A. I. Ismatul et al., Asian Journal of Biochemical and Pharmaceutical Research, 3, 90-98 (2012)], as has L. plantarum WCFS1 [M. H. J. Sturme et al., Microbiology, 153, 3939-3947 (2007)].

The L. plantarum strains were prepared and assayed in the same manner as Example 1 against three microorganisms Staphylococcus aureus, Enterococcus faecium and Escherichia coli. The OD₆₀₀ readings after 72 hours are presented in table 3. None of the cultures with L. plantarum strains reached an OD₆₀₀ reading of above 0.2 after 72 hours (the level for TTD in Example 1), but the OD₆₀₀ readings for L. plantarum CNCM I-4026 were significantly lower than those of the other L. plantarum strains ATCC 8014 and WCFS1. This demonstrates that L. plantarum CNCM I-4026 is particularly effective in limiting the growth of microorganisms including Staphylococcus aureus, an important cause of skin infections.

TABLE 3
OD₆₀₀ inhibition assay results for different L. plantarum strains after 72 hours

| | | | | MRS | |
|---|---|---|---|---|---|
| | | | | (Unfermented | |
| | NCC 2936 | WCFS1 | ATCC 8014 | broth) | Control |
| Staphylococcus aureus | 0.08 | 0.12 | 0.12 | 1.5 | 1.4 |
| Enterococcus faecium | 0.09 | 0.14 | 0.14 | 0.97 | 0.5 |
| Escherichia coli | 0.06 | 0.15 | 0.14 | 0.9 | 0.94 |

### Example 3: Oil-In-Water Emulsion Skin Cream Comprising a Bacterial Preparation of Lactobacillus plantarum CNCM I-4026

A sample of the freeze-dried supernatant of L. plantarum CNCM I-4026 from Example 1 is used to formulate a skin cream as follows:

Heat oil phase containing (in weight % overall):

Almond Oil 5%

Triglyceride 5%

Cetyl alcohol 2%

Stearic acid 2%

then blend with water phase containing:

Water 80%

2% freeze-dried Lactobacillus plantarum NCC2936 supernatant

Glycerin 2.5%

EDTA 0.5%

and finally add 1% paraben mix.

1. A method for the treatment or prevention of skin infections comprising administering a bacterial preparation of Lactobacillus plantarum CNCM I-4026 to an individual in need of same.

2. A method according to claim 1 wherein the bacterial preparation is applied topically.

3. A method according to claim 1 wherein the bacterial preparation is a fermentation broth of Lactobacillus plantarum CNCM I-4026 or an extract thereof.

4. A method according to claim 3 wherein the Lactobacillus plantarum CNCM I-4026 have been at least partially removed from the fermentation broth.

5. A method according to claim 1 wherein the Lactobacillus plantarum CNCM I-4026 have been rendered non-viable.

6. A method according to claim 1 wherein the bacterial preparation is the supernatant of a fermentation broth of Lactobacillus plantarum CNCM I-4026 after centrifugation.

7. A method according to claim 1 wherein the skin infections are selected from the group consisting of boils, carbuncles, styes, impetigo, cellulitis, acne vulgaris and combinations of these.

8. A method according to claim 1 wherein the skin infections are staphylococcal infections.

9. A method according to claim 1 wherein the skin infections are methicillin-resistant Staphylococcus aureus infections.

10. A method according to claim 1 wherein the composition is applied at a dosage of between 1 and 200 mg of the bacterial preparation of Lactobacillus plantarum CNCM I-4026 on a dry basis per 100 cm² of skin, 1 to 4 times per day.

11. A composition for use method according to claim 1 wherein the composition is administered to a subject suffering from burns, cuts, abrasions, pressure ulcers or combinations of these.

12. A method according to claim 1 wherein the composition is administered to a subject suffering from atopic dermatitis.

13. A method according to claim 1 wherein the composition further comprises a compound with antimicrobial properties selected from the group consisting of tea tree oil, eugenol, thymol, carvacrol, cinnamaldehyde, allyl isothiocyanate, lauric acid, palmitoleic acid, linoleic acid, acetic acid, propionic acid, butyric acid and combinations of these.

14. A method according to claim 1 wherein the composition is a pet care product.

15. A cosmetic composition comprising a bacterial preparation of Lactobacillus plantarum CNCM I-4026 wherein the composition is selected from the group consisting of cleansing, protective or care creams, milks or lotions for the face, for the hands, for the feet, for the major anatomical folds or for the body; makeup products; skincare gels or foams; shampoos, soaps; bath compositions; deodorant compositions, aftershave gels or lotions; and depilatory creams.
